(19) United States
(12) Patent Application Publication
(10) Pub. No.: US 2025/0091017 A1
(43) Pub. Date: Mar. 20, 2025

(54) TUNABLE HYDROPHILIC CROSSLINKED POLYMER MEMBRANES FOR SEPARATION APPLICATIONS

(71) Applicants: Georges Belfort, North Falmouth, MA (US); Pravav Ramesh, Troy, NY (US); RENSSELAER POLYTECHNIC INSTITUTE, Troy, NY (US)

(72) Inventors: Georges BELFORT, North Falmouth, MA (US); Pranav Ramesh, Troy, NY (US); Surya Karla, Troy, NY (US); James Kilduff, Saratoga Springs, NY (US)

(73) Assignee: RENSSELAER POLYTECHNIC INSTITUTE, Troy, NY (US)

(21) Appl. No.: 18/934,768

(22) Filed: Nov. 1, 2024

Related U.S. Application Data
(63) Continuation-in-part of application No. PCT/US2024/012895, filed on Jan. 25, 2024.
(60) Provisional application No. 63/624,866, filed on Jan. 25, 2024.
(60) Provisional application No. 63/441,051, filed on Jan. 25, 2023.
(60) Provisional application No. 63/547,231, filed on Nov. 3, 2023.

(51) Int. Cl. B01D 67/00; B01D 69/02; B01D 69/10; B01D 71/40; C07C 7/144; C08J 5/18; C08J 7/16

(52) U.S. Cl. CPC B01D 67/00931; B01D 67/0013; B01D 69/02; B01D 69/107; B01D 71/401; C07C 7/144; C08J 5/18; C08J 7/16; B01D 2323/02; B01D 2323/12; B01D 2323/21834; B01D 2323/30; B01D 2323/38; B01D 2325/36

(57) ABSTRACT

A membrane for separating organic solvents such as methanol and toluene is provided. A plurality methacrylate polymer brushes, e.g., composed of hydroxyethyl methacrylate (HEMA) monomers or aminoethyl methacrylate (AEMA) monomers, are grafted from a crosslinked polyimide support using Single Electron Transfer-Living Radical Polymerization (SET-LRP). The polymer brushes themselves are also crosslinked by ethylene glycol dimethacrylate (EGDMA), triethylene glycol dimethacryalte (TEGDMA) trimesic acid, and/or itaconic acid. These hydrophilic polymeric brush membranes demonstrate pore stiffening and yet also opening, obtaining high selectivity at reasonable permeability and reduced energy requirements for commercially relevant separations, e.g., methanol/toluene. The addition of the crosslinker prevents loss of selectivity as a result of imparting increased rigidity, enabling the membranes to be operated at higher operating pressures for increased throughput. These membranes would be beneficial for use in pharmaceutical, chemical, petroleum, food, and biotechnology industries, e.g., in the manufacture of polymethacrylic acid, the manufacture of paraxylene, etc.

### CROSS-REFERENCE TO RELATED APPLICATIONS

This application is a continuation-in-part of International Patent Application No. PCT/US24/12895, filed Jan. 25, 2024, which claims the priority benefit of U.S. Provisional Patent Application Nos. 63/441,051, filed Jan. 25, 2023, and 63/624,866, filed Jan. 25, 2024, the entireties of which are explicitly incorporated by reference herein. This application also claims the benefit of U.S. Provisional Application No. 63/547,231, filed Nov. 3, 2023, which is incorporated by reference as if disclosed herein in its entirety.

### STATEMENT REGARDING FEDERALLY SPONSORED RESEARCH OR DEVELOPMENT

This invention was made with government support under Grant Number FG02-09ER16005 awarded by the Department of Energy. The government has certain rights in the invention.

### BACKGROUND

Membranes offer a rate-driven, modular, and economic pathway for separation and purification of compounds. Polymeric membranes have found large scale utilization in a variety of applications such as desalination of sea water and protein purification. Industrial separation of organic solvents with these membranes remains a challenge due to limitations of the component polymers such as pore flexibility, as well as lack of structure by design and stability of polymers in organic solvents. As a consequence, distillation, an energy intensive phase-change process, remains the preferred mode of separation/purification of organics. Replacement of distillation columns with membranes could allow for a ten-fold decrease in energy utilization.

Methanol and toluene are organic solvents and appear together in a variety of processes such as paraxylene synthesis. Industrial methods to recover these solvents involve the use of distillation. Separation using this technique is further complicated by formation of an azeotrope and hence extractive distillation is often used. Membrane applications for this separation have been limited to the use of pervaporation, a relatively difficult membrane process that uses vacuum.

### SUMMARY

Aspects of the present disclosure are directed to a membrane for separating organic solvents. In some embodiments, the membrane includes a support membrane, the support membrane composed of a polyimide, a polyimide copolymer, or combinations thereof. In some embodiments, the membrane includes a polymer brush layer including a plurality of polymer brushes grafted from a surface of the support membrane, the polymer brushes including hydroxyethyl methacrylate (HEMA) monomers, aminoethyl methacrylate (AEMA) monomers, or combinations thereof. In some embodiments, the polymer brush layer includes a plurality of crosslinks between the polymer brushes, wherein the crosslinks include one or more crosslinkers composed of ethylene glycol dimethacrylate (EGDMA), triethylene glycol dimethacrylate (TEGDMA), trimesic acid, itaconic acid, or combinations thereof. In some embodiments, the mol. % crosslinker in the polymer brush layer is between about 30% and about 70%. In some embodiments, the polymer brush layer has a thickness of about 5 nm. In some embodiments, the support membrane is crosslinked with diethylene triamine (DETA).

Aspects of the present disclosure are directed to a method of making a membrane for separating organic solvents. In some embodiments, the method includes preparing a casting solution, the casting solution including a concentration of a polyimide; casting, from the casting solution, a support membrane including the polyimide; and grafting a plurality of polymer brushes from a surface of the support membrane to form a polymer brush layer, the polymer brushes including HEMA monomers, AEMA monomers, or combinations thereof. In some embodiments, the method includes contacting the support membrane with diethylene triamine (DETA) to form crosslinks in the support membrane. In some embodiments, grafting a plurality of polymer brushes from a surface of the support membrane includes contacting the support membrane with a grafting solution in the presence of a catalyst, the grafting solution including a concentration of HEMA monomers, initiator, and ligand, wherein the initiator includes ethyl alpha bromo isobutyrate and the ligand includes pentamethyldiethylenetriamine (PMDETA).

In some embodiments, the grafting solution includes one or more crosslinkers. In some embodiments, the grafting solution includes between about 2% and about 10% mol. %. crosslinker. In some embodiments, the crosslinkers include EGDMA, TEGDMA, trimesic acid, itaconic acid, or combinations thereof.

In some embodiments, the casting solution includes between about 20 and about 25 wt. % polyimide. In some embodiments, the molar ratio of HEMA monomer, initiator, and ligand in the grafting solution are 200:1:1. In some embodiments, the catalyst is a copper plate.

Aspects of the present disclosure are directed to a method of separating organic solvents. In some embodiments, the method includes preparing a membrane including a crosslinked support membrane, the support membrane composed of a polyimide polymer, a polyimide copolymer, or combinations thereof; and polymer brush layer; and contacting a medium including two or more organic solvents with the membrane. In some embodiments, contacting a medium including two or more organic solvents with the membrane occurs at a pressure across the membrane greater than about 10 bar. In some embodiments, contacting a medium including two or more organic solvents with the membrane occurs at a pressure across the membrane of about 50 bar.

In some embodiments, the polymer brush layer includes a plurality of polymer brushes grafted from a surface of the support membrane, the polymer brushes including HEMA monomers, AEMA monomers, or combinations thereof, wherein the polymer brushes are a crosslinked network including a plurality of crosslinks, wherein the crosslinks are composed of EGDMA, TEGDMA, trimesic acid, itaconic acid, or combinations thereof. In some embodiments, the mol. % crosslinker in the polymer brush layer is between about 30% and about 70%. In some embodiments, the polymer brush layer has a thickness of about 5 nm. In some embodiments, the two or more organic solvents include a first solvent including methanol and a second solvent including toluene.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A is a schematic representation of a membrane for separating organic solvents according to some embodiments of the present disclosure;

FIG. 1B is a schematic representation of a membrane for separating organic solvents according to some embodiments of the present disclosure;

FIG. 2 is a chart of a method of making a membrane for separating organic solvents according to some embodiments of the present disclosure;

FIG. 3A is a graph portraying attenuated total reflectance for Fourier transform infrared (ATR-FTIR) spectra showing crosslinked polyimide supports (cPI) and 100 mol. % HEMA grafted cPI according to some embodiments of the present disclosure;

FIG. 3B is a graph portraying ATR-FTIR spectra showing various crosslinked HEMA grafted cPI according to some embodiments of the present disclosure;

FIG. 3C is a graph portraying ATR-FTIR spectra showing various crosslinked HEMA grafted cPI according to some embodiments of the present disclosure;

FIG. 3D is a graph portraying degree of grafting (DG) of various crosslinked HEMA grafted cPI according to some embodiments of the present disclosure;

FIG. 3E is a graph portraying percent crosslinker crosslinked HEMA grafted cPI according to some embodiments of the present disclosure as determined from x-ray photoelectron spectroscopy (XPS) measurements;

FIG. 3F is a graph portraying roughness as a function of crosslinker type and concentration of HEMA grafted cPI according to some embodiments of the present disclosure as measured by atomic force microscopy (AFM);

FIGS. 4A-4D are graphs portraying quartz crystal microbalance with dissipation (QCM-D) measurements of various HEMA grafted cPI according to some embodiments of the present disclosure;

FIG. 5 is a chart of a method of separating organic solvents according to some embodiments of the present disclosure;

FIG. 6A is a graph portraying solvent permeabilities for various HEMA grafted cPI according to some embodiments of the present disclosure;

FIG. 6B is a graph portraying permeabilities for methanol and toluene for various HEMA grafted cPI according to some embodiments of the present disclosure;

FIG. 6C is a graph portraying selectivities as a function of pressure for various HEMA grafted cPI according to some embodiments of the present disclosure; and

FIG. 6D is a graph portraying permeabilities as a function of pressure for various HEMA grafted cPI according to some embodiments of the present disclosure.

### DETAILED DESCRIPTION

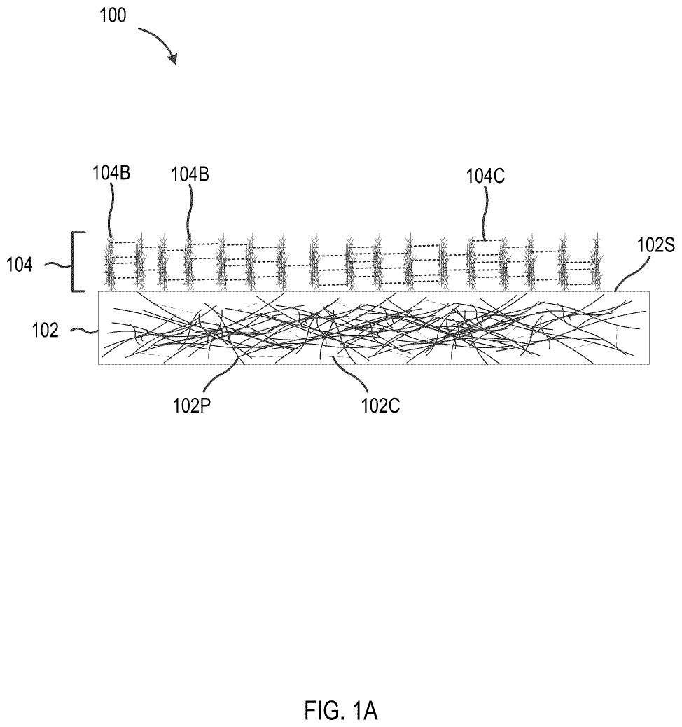

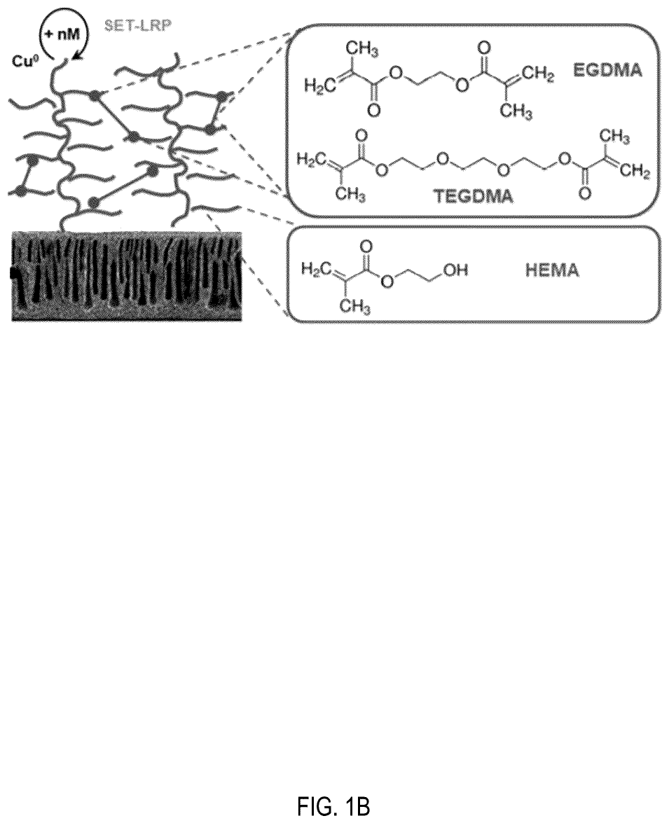

Referring now to FIG. 1, some embodiments of the present disclosure are directed to a membrane 100 for separating organic solvents, e.g., in a mixture of two or more organic solvents. In some embodiments, at least one of the organic solvents is an apolar organic solvent. Organic solvents for separation by exemplary embodiments of the present disclosure include, but not limited to, methanol, ethanol, isopropanol, isobutanol, butanol, toluene, triisopropyl benzene, etc., and combinations thereof. In some embodiments, the mixture of two or more organic solvents includes one or more dyes. In some embodiments, the one or more dyes has a molecular weight greater than about 350 g/mol.

In some embodiments, membrane 100 includes a support membrane 102. In some embodiments, support membrane 102 is any suitable shape, size, and thickness to facilitate the desired separation, as will be understood by those of skill in the art. In some embodiments, support membrane 102 is generally hydrophobic. In some embodiments, support membrane 102 is generally hydrophilic.

In some embodiments, support membrane 102 includes a network of one or more polymers 102P. In some embodiments, support membrane 102 is composed of a polyimide, a polyimide copolymer, or combinations thereof. In exemplary embodiments, support membrane 102 is composed of P84® polyimide (EVONIK FIBRES GMBH, Austria). In some embodiments, support membrane 102 includes a plurality of crosslinks 102C. In some embodiments, crosslinks 102C include one or more crosslinkers. In some embodiments, the crosslinkers in crosslinks 102C include diethylene triamine (DETA).

In some embodiments, membrane 100 includes a polymer brush layer 104. In some embodiments, polymer brush layer 104 is positioned on a surface 102S of support membrane 102. In some embodiments, polymer brush layer 104 is at least partially incorporated into support membrane 102 and extends outwardly therefrom. In some embodiments, polymer brush layer 104 includes a plurality of polymer brushes 104B. In some embodiments, polymer brushes 104B are grafted from surface 102S, as will be discussed in greater detail below. In some embodiments, polymer brush layer 104B has a thickness of about 5 nm.

In some embodiments, polymer brushes 104B are composed of one or more polymers. In some embodiments, each polymer brush 104B is composed of monomers having the same or substantially the same structure. In some embodiments, at least some polymer brushes 104B are copolymers. In some embodiments, In some embodiments, at least some polymer brushes 104B are block copolymers. In some embodiments, polymer brush layer 104 is composed of a first portion of polymer brushes 104B having a first composition and a second portion of polymer brushes 104B having a second composition. In some embodiments, the first composition and the second composition include different monomers. In some embodiments, at least some polymer brushes 104B include hydroxyethyl methacrylate (HEMA) monomers. In some embodiments, a majority of the monomers in polymer brushes 104B are HEMA, e.g., greater than 50 mol. %. In some embodiments, all monomers in polymer brushes 104B are HEMA. In some embodiments, at least some polymer brushes 104B include aminoethyl methacrylate (AEMA) monomers. In some embodiments, a majority of the monomers in polymer brushes 104B are AEMA, e.g., greater than 50 mol. %. In some embodiments, all monomers in polymer brushes 104B are AEMA.

In some embodiments, polymer brush layer 104 includes a plurality of crosslinks 104C between polymer brushes 104B. In some embodiments, crosslinks 104C include one or more crosslinkers. In some embodiments, the crosslinkers in crosslinks 104C include ethylene glycol dimethacrylate (EGDMA), triethylene glycol dimethacrylate (TEGDMA), trimesic acid, itaconic acid, or combinations thereof. In some embodiments, the mol. % crosslinker in polymer brush layer 104B is between about 30% and about 70%.

Referring now to FIG. 1B, polymer brushes 104B in polymer brush layer 104 provide improved selectivity and permeability to membrane 100. To address any selectivity-permeability trade-off, the brushes were stiffened using a Single Electron Transfer-Living Radical Polymerization (SET-LRP) reaction scheme, as will be discussed in greater detail below. In an exemplary embodiment, a support membrane of P84® polyimide was modified with graft polymerized hydrophilic brushes. In this exemplary embodiment, HEMA hydrophilic brushes with short EGDMA and long TEGDMA crosslinkers were used. Nature, in the form of aquaporin channels for selective water transport, uses mostly hydrophobic channel walls to minimize hydrogen-bond interactions, thereby freeing the water molecules for transport. In embodiments of the present disclosure, it was reasoned that apolar organic solvents exhibit less interactions with a pore surface formed from hydrophilic brushes and crosslinkers than from hydrophobic-walled pores-thus minimizing hydrophobic interactions and allowing the solvent molecules to transport more freely.

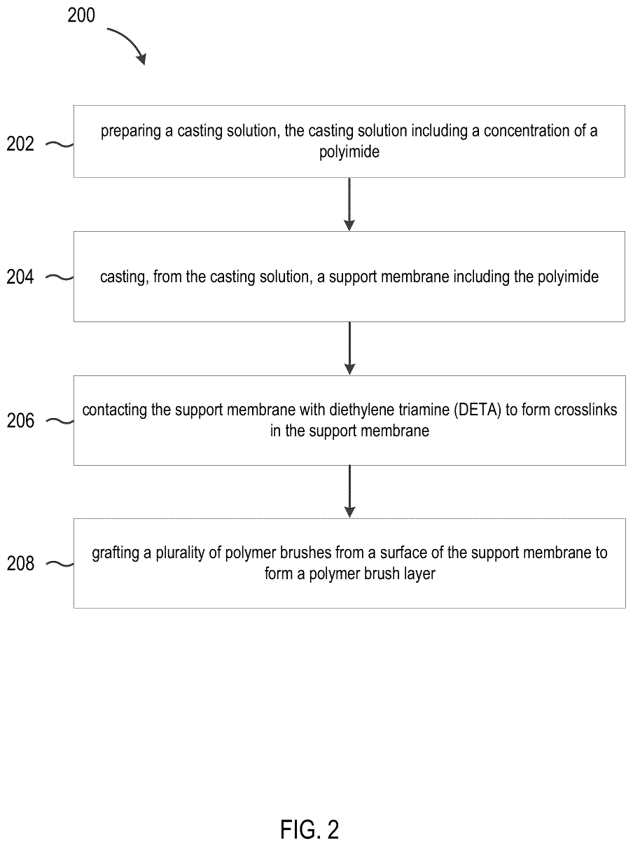

Referring now to FIG. 2, some embodiments of the present disclosure are directed to a method 200 of making a membrane for separating organic solvents. As discussed above, organic solvents for separation by exemplary embodiments of the present disclosure, e.g., membranes 100 made by method 200, include but are not limited to, methanol, ethanol, isopropanol, isobutanol, butanol, toluene, triisopropyl benzene, etc., or combinations thereof. At 202, a casting solution is prepared. In some embodiments, the casting solution includes a concentration of monomers, oligomers, etc. desired to form a support membrane. In some embodiments, the casting solution includes a concentration of a polyimide, e.g., polyimide monomers, oligomers, etc., or combinations thereof. In some embodiments, the casting solution includes between about 20 and about 25 wt. % polyimide. In some embodiments, the casting solution includes about 24 wt. % polyimide. At 204, a support membrane, e.g., including the polyimide, is cast from the casting solution. In some embodiments, at 206, the support membrane is contacted with DETA to form crosslinks in the support membrane.

Still referring to FIG. 2, at 208 a plurality of polymer brushes are grafted from a surface of the support membrane to form a polymer brush layer. As discussed above, in some embodiments, the polymer brushes include HEMA monomers, AEMA monomers, or combinations thereof.

In an exemplary embodiment, grafting 208 a plurality of polymer brushes from a surface of the support membrane is performed using SET-LRP. In some embodiments, grafting 208 includes contacting the support membrane with a grafting solution in the presence of a catalyst. In some embodiments, the grafting solution includes a concentration of monomers, initiator, and ligand. In some embodiments, the molar ratio of monomer, initiator, and ligand in the grafting solution are 200:1:1. In some embodiments, the grafting solution includes a concentration of HEMA monomers, initiator, and ligand. In some embodiments, the molar ratio of HEMA monomer, initiator, and ligand in the grafting solution are 200:1:1. In some embodiments, the initiator includes ethyl alpha bromo isobutyrate. In some embodiments, the ligand includes pentamethyldiethylenetriamine (PMDETA). In some embodiments, the catalyst includes copper. In some embodiments, the grafting solution is in contact with a copper plate as the catalyst. The copper plate, in addition to catalyzing the grafting of polymer brushes from the support membrane, can help to keep the support membrane and polymer brush layer substantially flat. In some embodiments, the grafting reaction from step 208 is allowed to continue for at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, or 16 hours.

In some embodiments, the grafting solution includes one or more crosslinkers. As discussed above, in some embodiments, the crosslinkers include EGDMA, TEGDMA, trimesic acid, itaconic acid, or combinations thereof. In some embodiments, the grafting solution includes between about 2% and about 10% mol. % crosslinker. The graft polymerization is carried out with SET-LRP that offers a simple, green, and highly reproducible technique to form polymers with a low polydispersity index.

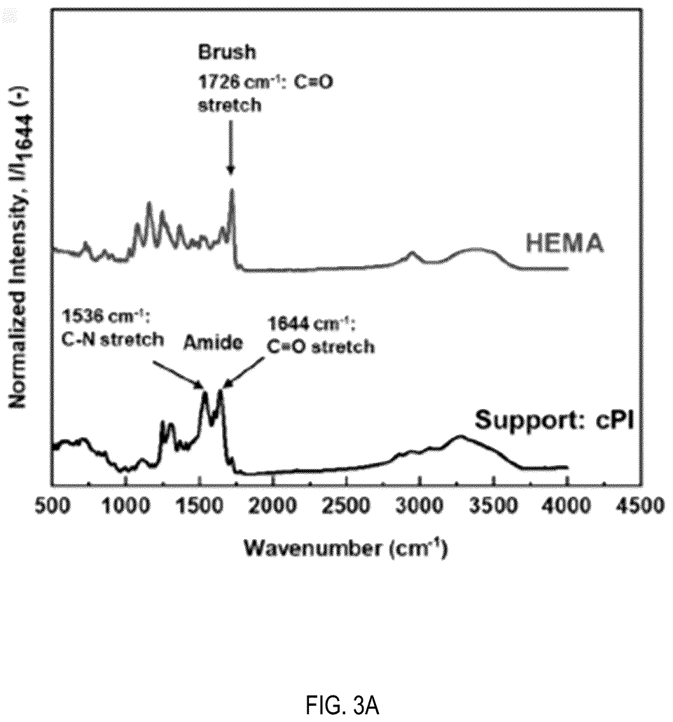

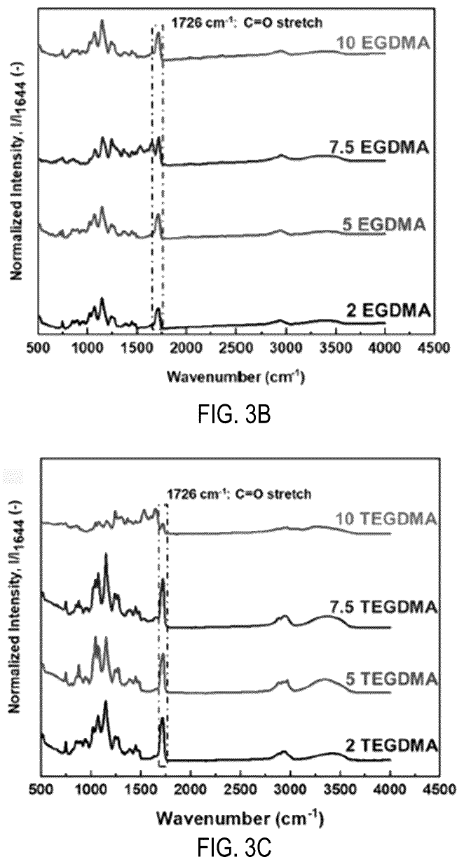

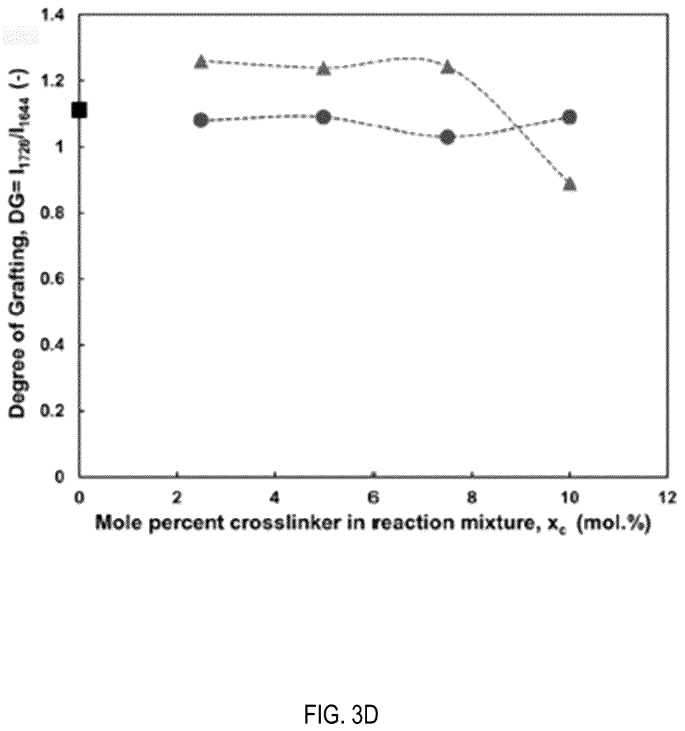

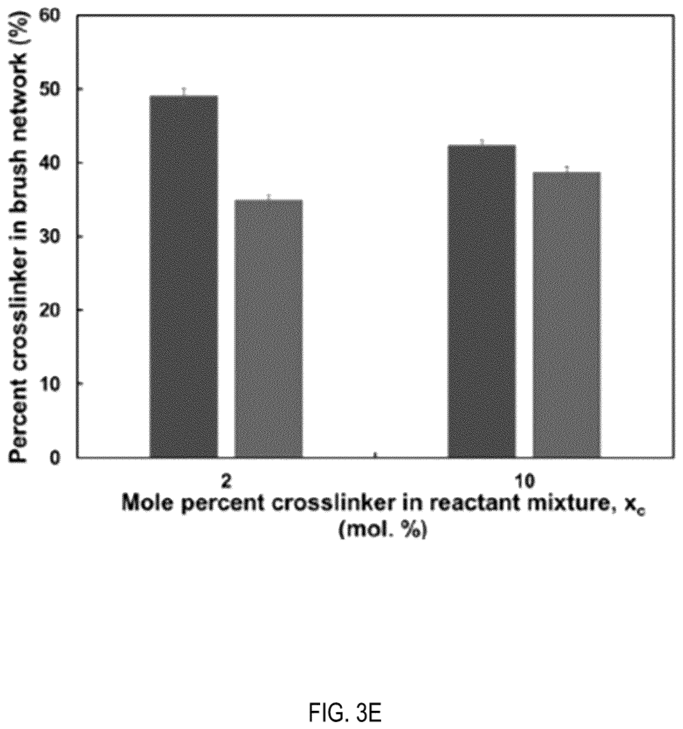

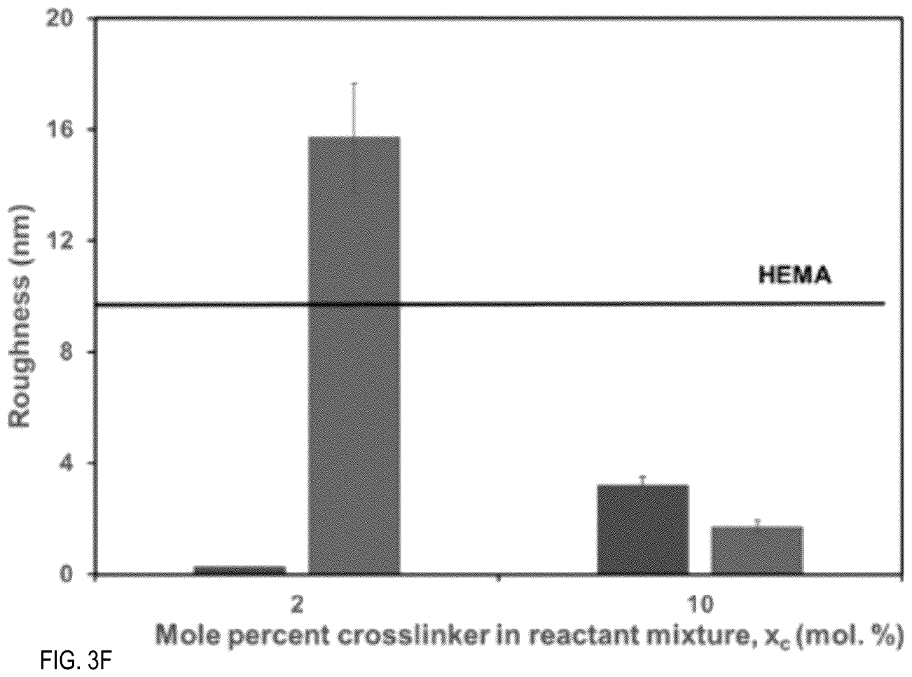

Referring now to FIGS. 3A-3F, attenuated total reflectance for Fourier transform infrared (ATR-FTIR) spectra are provided of an exemplary membranes including crosslinked polyimide (cPI) and HEMA-modified cPI membranes. These spectra confirm successful modification of the support as seen by an increase in the peak at 1726 cm⁻¹ and 2922 cm⁻¹ attributed to the stretch of the ester and hydrocarbon groups from the graft polymer/crosslinker, respectively. There was also a change in the nature of the peak at 3500 cm⁻¹ due to the addition of HEMA onto the surface including hydroxyl bending modes. Individual absorbances were normalized to internal standards with peaks 1644 cm⁻¹ and 1535 cm⁻¹, corresponding to the C═O and C—N stretch of amide groups from the support cPI.

Referring specifically to FIGS. 3B and 3C, crosslinking of the HEMA-grafted brushes on the support membrane was carried out using shorter EGDMA or longer TEGDMA at 4 different concentrations: 2, 5, 7.5, and 10 mol. % crosslinker. It is apparent that there is a change in intensity of the ester stretch peak at 1726 cm⁻¹ as a result of addition of the crosslinker. This change can be quantified by defining a degree of grafting, DG=11726/11644 that allows for comparison of the effect of crosslinker concentration on surface modification.

Referring now to FIG. 3D, the variation in degree of grafting (DG) is shown as a function of crosslinker concentration. The DG values obtained for TEGDMA (triangles) are greater than those with EGDMA (circles). However, DGs with crosslinkers lie in the region of the control (black). Structurally, TEGDMA and EGDMA include the same number of ester bonds per molecule. Additionally, the intensity obtained in ATR-FTIR was affected by electron shielding and hence it is prudent to restrict DG comparisons within a monomer type. For EGDMA, the DG remained fairly constant with increasing crosslinker concentration. In contrast, the DG for TEGDMA remained constant up to 7.5 mol. % followed by a rapid decline. Without wishing to be bound by theory, since TEGDMA is a significantly bulkier molecule than HEMA, the presence of this crosslinker may interfere with HEMA graft polymerizing on the surface. Estimation of DG using the amide peak at 1535 cm⁻¹ yielded similar values.

Referring now to FIG. 3E, graft-modifications were also performed on silica wafers to obtain complementary information from x-ray photoelectron spectroscopy (XPS) measurements regarding the chemical nature of surface modification, with XPS used to distinguish between crosslinkers and HEMA control. The mol. % crosslinker and individual component concentration values are summarized in FIG. 3E. Due to the addition of the crosslinkers EGDMA and TEGDMA, less HEMA was grafted on the surface. However, the amount of crosslinker in the brush network remains relatively constant. From ATR-FTIR and XPS characterization, the total amount of C═O on the surface was relatively constant. However, the amount of C—C groups on the surface increased. Without wishing to be bound by theory, this can be attributed to the longer chain length of the crosslinked brushes as opposed to just HEMA.

Referring now to FIG. 3F, the roughness of the grafted brush layers on silica was measured with atomic force microscopy (AFM). Static contact angles of the crosslinked and unmodified brushes did not change significantly (57.4±3.80) with increasing mol percent monomers from 0-10 mol. % in solution. Unmodified silica (control) appears smooth with a root mean square (RMS) roughness of 0.14 nm. HEMA in the absence of crosslinker showed increased roughness of 9.74 nm compared with the crosslinker. The roughness of the surface modified with the short crosslinker (EGDMA) increased with increasing crosslinker concentration. The reverse is observed for the longer crosslinker (TEGDMA) where roughness decreases with increasing crosslinker concentration. Without wishing to be bound by theory, a decrease in the roughness could have been due to uniform grafting and/or the absence of grafting on the surface as the bare silica was smooth. At small concentrations of EGDMA linker, the presence of the crosslinker may have allowed for more uniform HEMA grafting onto the surface. A large number of HEMA molecules were hindered from surface grafting due to stearic hinderance with the longer crosslinker. This may explain why the roughness of HEMA without crosslinker is greater.

It is apparent that at low EGDMA concentrations, the surface is extremely smooth while at larger concentrations of the same crosslinker, areas with increasing roughness are observable. With increasing crosslinker concentration, the crosslinker competed with HEMA for attachment and growth on the surface leading to increased roughness. The longer crosslinker on the other hand, at low concentrations, prevented the grafting of HEMA on the surface leading to non-uniform grafting. The TEGDMA can also attach and polymerize and lead to elevated roughness. At increasing concentrations of the long crosslinker, surface sites are lost leading to a lower roughness. This is in good agreement with DG values obtained from ATR-FTIR wherein DG decreases with concentration for the long crosslinker but remains relatively uniform for the short crosslinker. AFM measurements offered proof that HEMA grafting on the surface was influenced by the size of the crosslinker.

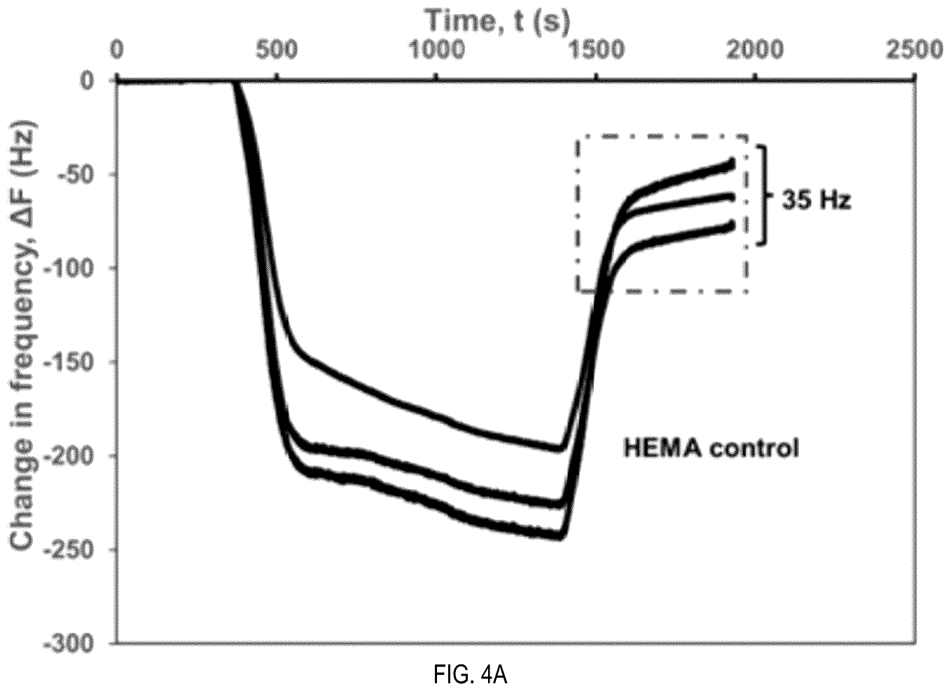

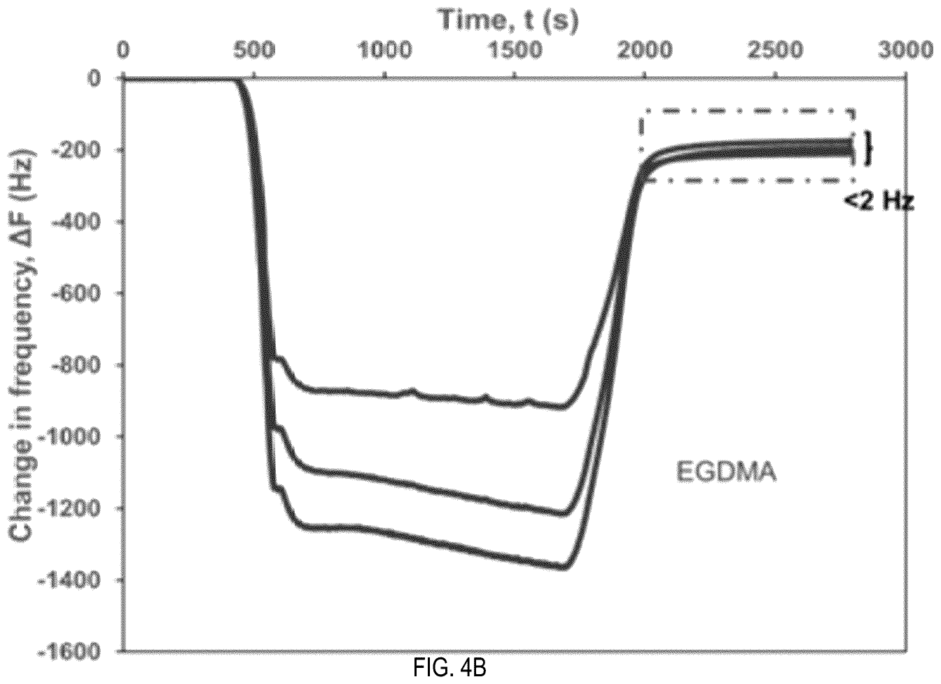

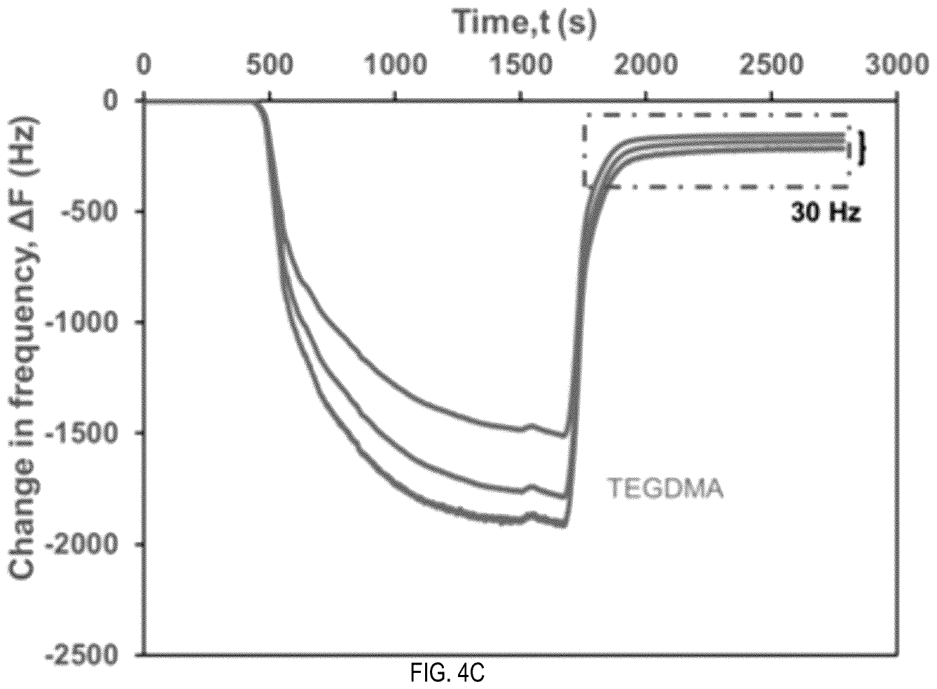

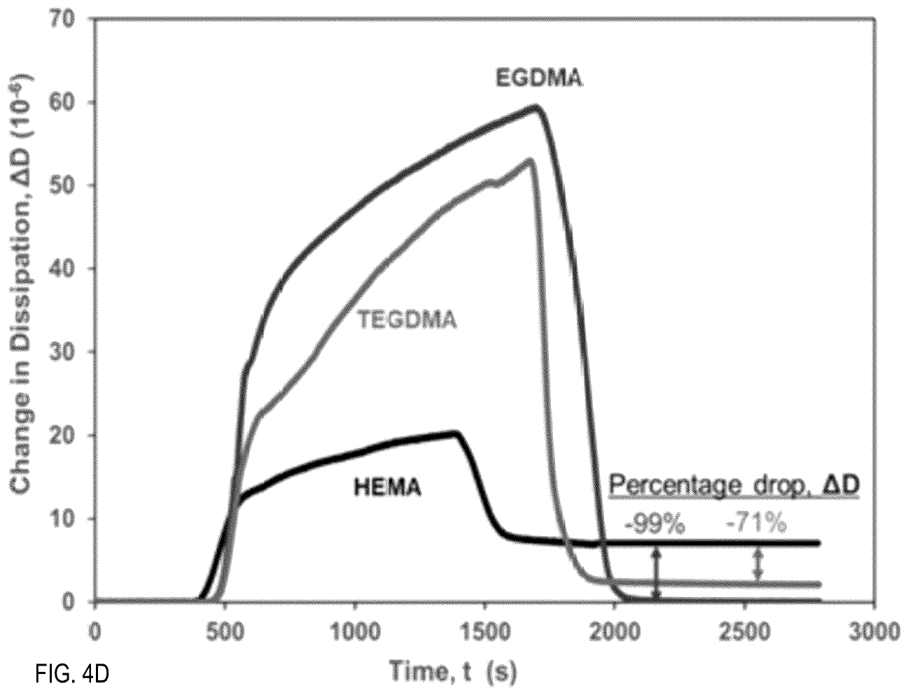

Referring now to FIGS. 4A-4D, the stiffness of the grafted brushes layer was studied using quartz crystal microbalance with dissipation (QCM-D) to determine the effect of crosslinking on the rigidity of the brush network for pHEMA and crosslinked pEGDMA and pTEGDMA, respectively, so as to correlate it with membrane filtration performance. Both the frequency and the dissipation data enabled the rigidity comparison directly, as lower changes in frequency (ΔF) overtone values spread less and lower dissipation (ΔD) values correlate with increased rigidity. At the end of one cycle of modification and washing, the ΔF overtone spread values were approximately 35, <2, and 30 Hz for the HEMA, EGDMA and TEGDMA crosslinked membranes, respectively, indicating that crosslinking with the shorter crosslinker EGDMA strongly rigidifies the brush membranes, while grafting with the longer crosslinker TEGDMA induces a similar but less rigid effect.

Referring now specifically to FIG. 4D, the results for ΔD values exhibited a 99% and 71% decrease in dissipation compared with the non-crosslinked HEMA case, as a result of crosslinking with EGDMA and TEGDMA, respectively. To account for mass differences, a normalized ΔD/ΔF ratio offers a tool for comparison where ΔD/ΔF values less than 10⁻⁸ are representative of rigid layers. The presence of the crosslinker resulted in ΔD/ΔF values of 1.8×10⁻¹⁰ Hz and 1.3×10⁻⁸ Hz for EGDMA and TEGDMA, respectively, as opposed to 17×10⁻⁸ Hz in the absence of crosslinker. Performing viscoelastic modeling and utilizing Voigt fits for HEMA and crosslinker (EGDMA) allowed the estimation of thickness of the grafted layer (nm), the elastic modulus (kPa) and the viscosity (Pas) by minimizing the error function (χ2). Values of brush thickness were determined to be 5 nm for HEMA and EGDMA. An order of magnitude increase in elastic modulus, 150 kPa to 1750 kPa, and a three-fold increase in viscosity, 1000 Pa s to 3000 Pa s, were observed for HEMA and EGDMA crosslinked HEMA, respectively. Values of ΔF and ΔD results discussed above indicate that the presence of this crosslinker led to an increase in the rigidity of the grafted brush network.

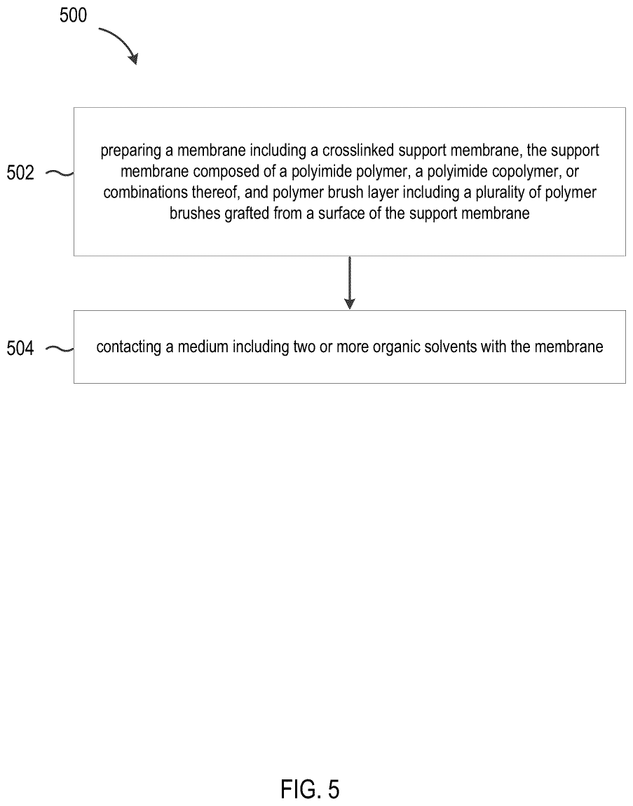

Referring now to FIG. 5, some embodiments of the present disclosure are directed to a method 500 of separating organic solvents. At 502, a membrane for separating the organic solvents is prepared, e.g., via method 200 described above. In some embodiments, the membrane includes a crosslinked support membrane composed of a polyimide polymer, a polyimide copolymer, or combinations thereof. In some embodiments, the membrane includes a polymer brush layer that includes a plurality of HEMA polymer brushes grafted from a surface of the support membrane. In some embodiments, the HEMA polymer brushes are a crosslinked network including a plurality of crosslinks composed of EGDMA, TEGDMA, or combinations thereof. As discussed above, in some embodiments, the mol. % crosslinker in the polymer brush layer is between about 30% and about 70%. In some embodiments, the polymer brush layer has a thickness of about 5 nm. In some embodiments, the membrane includes a polymer brush layer that includes a plurality of AEMA polymer brushes grafted from a surface of the support membrane. In some embodiments, the AEMA polymer brushes are a crosslinked network including a plurality of crosslinks composed of trimesic acid, itaconic acid, or combinations thereof.

At 504, a medium including two or more organic solvents is contacted with the membrane. In some embodiments, pressure is applied across the membrane, driving transport of a desired solvent across the membrane and facilitating separation of at least one solvent from the remainder of the medium. In some embodiments, the pressure applied across the membrane is greater than about 10, 20, 30, or 40 bar. In some embodiments, the pressure applied across the membrane is 50 bar. In some embodiments, the pressure applied during step 504 is substantially constant. In some embodiments, the pressure applied during step 504 is variable, and on average greater than about 10, 20, 30, or 40 bar. In some embodiments, the pressure applied during step 504 is variable, and on average about 50 bar. As discussed above, in some embodiments, the medium includes methanol, ethanol, isopropanol, isobutanol, butanol, toluene, triisopropyl benzene, etc., or combinations thereof. In some embodiments, the two or more organic solvents in the medium include a first solvent including methanol and a second solvent including toluene.

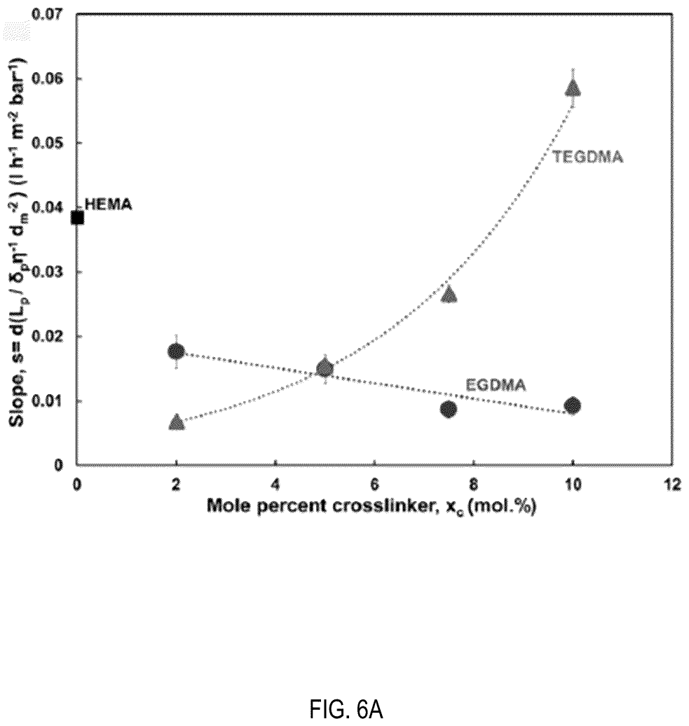

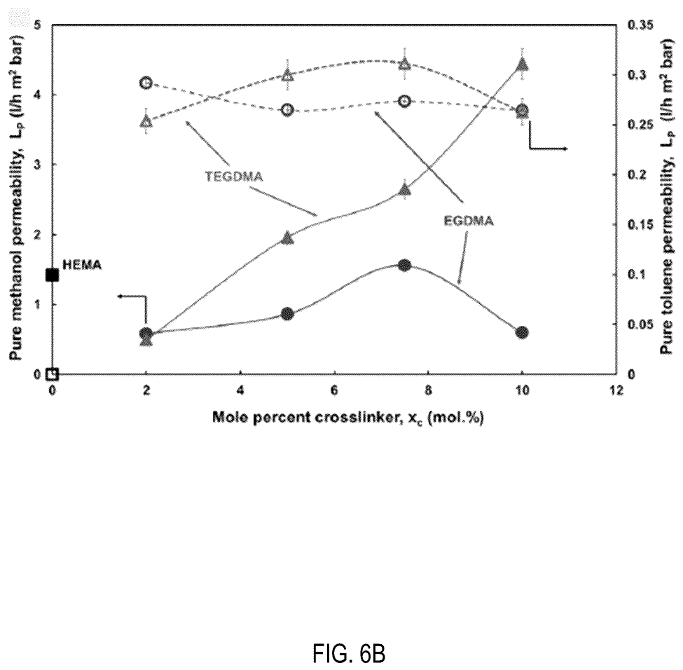

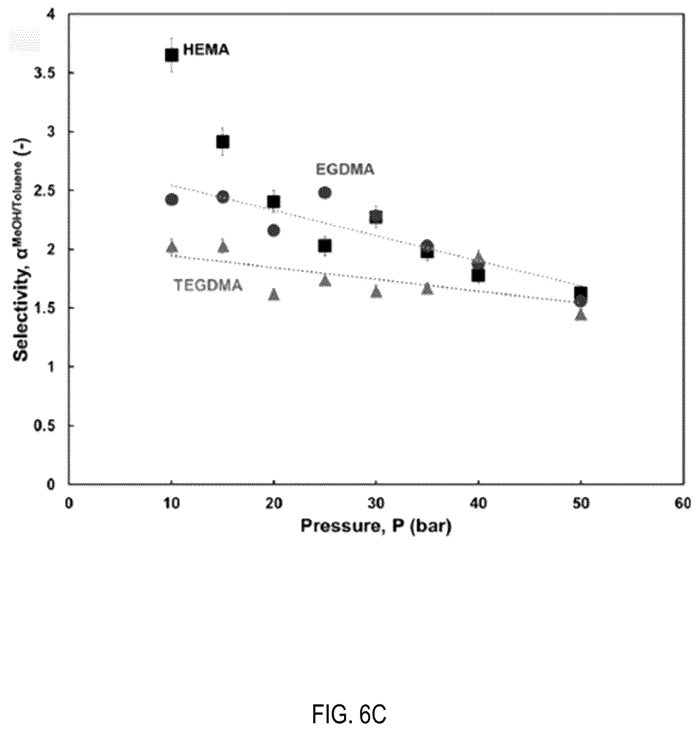

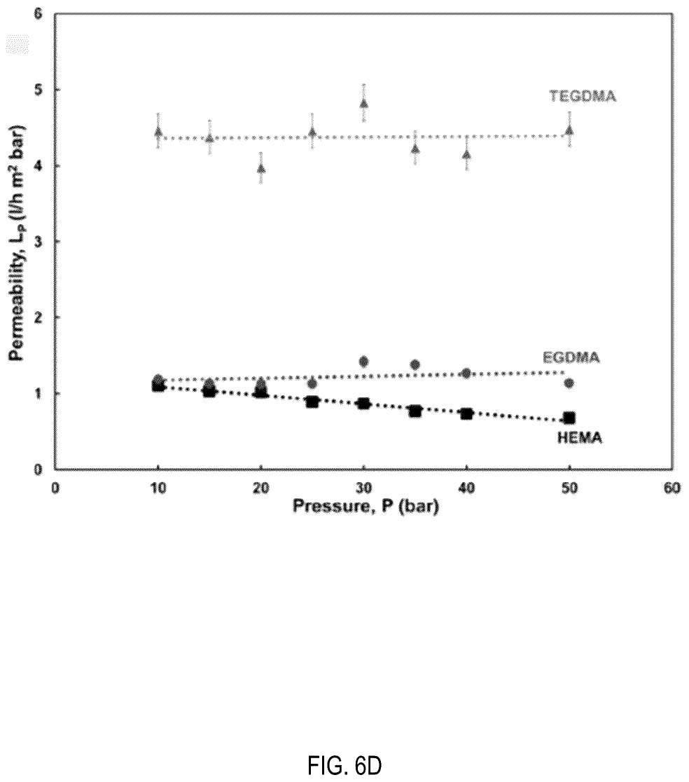

Referring now to FIG. 6A, the effect of solvent properties on membrane permeability were measured using water and a series of alcohols, including methanol, ethanol, isopropanol, butanol, isobutanol, and octanol. The permeability of these solvents through the membranes was proportional to three solvent parameters: Hansen solubility, molar volume, and kinematic viscosity, and followed a linear trend. The slopes from these linear curves offer insight into the effect of crosslinking on permeability and viscosity. Increasing crosslinker concentration led to an exponential increase in the slope for the long crosslinker pTEGDMA. At highest crosslinker concentration, the slope was greater than that of the control pHEMA (no crosslinker). On the other hand, the slope for the shorter crosslinker pEGDMA decreased linearly with crosslinker concentration. The XPS analysis discussed above showed that about 2-10 times the amount of pEGDMA than pTEGDMA was grafted in the pHEMA brush structure, indicating a looser structure with increased flux for the longer crosslinker than the shorter one. This is confirmed with methanol pure flux results (see FIG. 6B) where the flux was higher and increased with TEGDMA crosslinker as compared with the lower and relatively constant flux with EGDMA crosslinker. pHEMA with no crosslinker exhibited zero toluene permeation. However, pure toluene fluxes were relatively constant and similar in value for both crosslinkers with increasing crosslinker concentration.

Referring now to FIG. 6B, brush membranes comprising pHEMA and pEGDMA (2 and 10 mol. %) and pHEMA and pTEGDMA (2 and 10 mol. %) were tested for their ability to separate methanol-toluene mixtures using a dead-end filtration cell. Results with mixtures of 90-10 mol. % MeOH-toluene, simulating typical ratios used in industry, exhibited selectivity of 3.7±0.2 with HEMA and lower values with crosslinked EGDMA or TEGDMA brushes, and further reduced with increasing operating pressure.

The methanol flux of EGDMA demonstrates an increase with increase in crosslinker concentration followed by a fall. However, this change is minimal compared to the longer crosslinker TEGDMA wherein the permeability increases with increasing crosslinker concentration. This increase is almost 10× for 10 mol. % crosslinker compared with 2 mol. % crosslinker. Without wishing to be bound by theory, this can be attributed to the more open brush network of TEGDMA which allows for increased pathways for MeOH transport.

Referring now to FIG. 6C, membranes modified with crosslinkers EGDMA (2 and 10 mol. %) lead to lower selectivity around 1.5 and 2, respectively, and those with TEGDMA (2 and 10 mol. %) show selectivity around 1.5. For all 3 membranes (HEMA, and crosslinked with EGDMA and TEGDMA), the methanol permeation flux and its concentration in the permeate decreased with increasing pressure. This is attributed to membrane concentration polarization of toluene inhibiting the permeation of the smaller solute, methanol, i.e., as pressure increased, concentration polarization increased allowing toluene concentration to grow at the interface on the feed side and hence block the methanol permeation flux. Compaction which increases in membrane resistance to permeate flow should have the same effect. It is apparent that pHEMA showed the best selectivity of 3.8 at 10 bar. However, as the pressure increases, the selectivity drops to 1.5 at 50 bar. The pHEMA with no crosslinks appeared to be particularly susceptible to this loss in selectivity at high pressures. The membranes with pEGDMA demonstrated a selectivity of 2.5 at 10 bar that dropped to 1.7 at 50 bar. pTEGDMA offered the lowest selectivity at 10 bar but appeared resistant to the effect of pressure. This is likely due to the increased stiffness from the crosslinked brushes.

Referring now to FIG. 6D, the permeability of these membranes for methanol-toluene mixtures was compiled as a function of pressure. Crosslinking the brushes led to an increase in permeability which is highest in the case of the long crosslinker TEGDMA where the permeability was around 4× greater than that of HEMA. The selectivity of these membranes was nearly the same at 30 bar. Therefore, crosslinking brushes allows for increased capacity (up to 550% increase) by allowing operation at higher feed pressures. The presence of the crosslinker reduces the selectivity by altering the brush network thereby offering channels for larger molecules to pass through. This effect is most pronounced with the larger crosslinker. However, the presence of the crosslinker reduces the effect of pressure on the performance of these membranes as a result of increased rigidity.

The synthetic polymeric nanofiltration membranes and processes consistent with embodiments of the present disclosure offer an alternate low-energy option to energy-intensive distillation to fractionate similar size organic solvents. These structure-by-design hydrophilic polymeric brush membranes address limitations of commercial polymer membranes and are tunable and exhibit commercially relevant filtration performance. In exemplary embodiments, graft polymerization using SET-LRP deposited HEMA, which were crosslinked with EGDMA and/or TEGDMA, on polyimide support membranes. These embodiments replace statistically random phase inversion or interfacial polymerization used for synthesizing commercial polymer membranes, and their porous structure can be remodeled by varying their morphology and chemistry. These hydrophilic polymeric brush membranes demonstrate pore stiffening and yet also opening, obtaining high selectivity at reasonable permeability and reduced energy requirements for commercially relevant separations, e.g., methanol/toluene.

The surface modifications performed on crosslinked polyimide supports consistent with embodiments of the present disclosure enabled the creation of a class of bottlebrush membranes with tunable organic transport properties. The synthesized membranes/surfaces were characterized using ATR-FTIR, XPS, QCM-D and contact angle measurements. Crosslinking of the brushes led to increased rigidity. The stability and performance of these membranes were tested using a series of pure solvents and an industrially significant solvent mixture (methanol-toluene). The synthesized crosslinked brush membranes were stable in these mediums, and further were able to effectively separate methanol-toluene mixtures with tunable performance.

Areas where these membranes may find application is for pharmaceutical, chemical, petroleum, food, and biotechnology industries, e.g., in the manufacture of polymethacrylic acid, the manufacture of paraxylene, etc. Separation of mixtures in these industries can be challenging because of the close molecular size of components, the formation of minimum azeotropes, etc. This typically means the use of complicated and often costly processes like adsorption, pervaporation or extractive distillation to obtain satisfactory separation.

Current methods of methanol-toluene separations involve distillation, extractive distillation, or adsorption which are energy intensive and costly. Membranes consistent with embodiments of the present disclosure offer a selectivity of ˜4 for this mixture separation, on par with selectivity for organic solvent nanofiltration membranes, and the permeabilities are an order of magnitude lower compared with hydrocarbon feed mixtures. The addition of the crosslinker prevents loss of selectivity as a result of imparting increased rigidity which was particularly valuable at higher operating pressures. These crosslinked brush membranes offer a route to separation of organic solvents using membrane filtration. When the crosslinkers provide sufficient selectivity, they will also impart a substantial increase in capacity of a membrane plant due to their significantly higher permeabilities. This trade-off provided by the different crosslinkers provides plant operators the flexibility to produce a desired product at desirable purity and productivity.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A membrane for separating organic solvents, comprising:
a support membrane, the support membrane composed of a polyimide, a polyimide copolymer, or combinations thereof; and
a polymer brush layer including a plurality of polymer brushes grafted from a surface of the support membrane, the polymer brushes including hydroxyethyl methacrylate (HEMA) monomers, aminoethyl methacrylate (AEMA) monomers, or combinations thereof.

2. The membrane according to claim 1, wherein the polymer brush layer includes a plurality of crosslinks between the polymer brushes, wherein the crosslinks include one or more crosslinkers composed of ethylene glycol dimethacrylate (EGDMA), triethylene glycol dimethacrylate (TEGDMA), trimesic acid, itaconic acid, or combinations thereof.

3. The membrane according to claim 2, wherein the mol. % crosslinker in the polymer brush layer is between about 30% and about 70%.

4. The membrane according to claim 2, wherein the polymer brush layer has a thickness of about 5 nm.

5. The membrane according to claim 1, wherein the support membrane is crosslinked with diethylene triamine (DETA).

6. A method of making a membrane for separating organic solvents, comprising:
preparing a casting solution, the casting solution including a concentration of a polyimide;
casting, from the casting solution, a support membrane including the polyimide; and
grafting a plurality of polymer brushes from a surface of the support membrane to form a polymer brush layer, the polymer brushes including hydroxyethyl methacrylate (HEMA) monomers, aminoethyl methacrylate (AEMA) monomers, or combinations thereof.

7. The method according to claim 6, wherein the casting solution includes between about 20 and about 25 wt. % polyimide.

8. The method according to claim 6, further comprising:
contacting the support membrane with diethylene triamine (DETA) to form crosslinks in the support membrane.

9. The method according to claim 6, wherein grafting a plurality of polymer brushes from a surface of the support membrane includes:
contacting the support membrane with a grafting solution in the presence of a catalyst, the grafting solution including a concentration of HEMA monomers, initiator, and ligand, wherein the initiator includes ethyl alpha bromo isobutyrate and the ligand includes pentamethyldiethylenetriamine (PMDETA).

10. The method according to claim 9, wherein the molar ratio of HEMA monomer, initiator, and ligand in the grafting solution are 200:1:1.

11. The method according to claim 9, wherein the grafting solution includes one or more crosslinkers.

12. The method according to claim 11, wherein the grafting solution includes between about 2% and about 10% mol. %. crosslinker.

13. The method according to claim 11, wherein the crosslinkers include ethylene glycol dimethacrylate (EGDMA), triethylene glycol dimethacrylate (TEGDMA), trimesic acid, itaconic acid, or combinations thereof.

14. The method according to claim 9, wherein the catalyst is a copper plate.

15. A method of separating organic solvents, comprising:
preparing a membrane including:
a crosslinked support membrane, the support membrane composed of a polyimide polymer, a polyimide copolymer, or combinations thereof; and
polymer brush layer including:
a plurality of polymer brushes grafted from a surface of the support membrane, the polymer brushes including hydroxyethyl methacrylate (HEMA) monomers, aminoethyl methacrylate (AEMA) monomers, or combinations thereof,
wherein the polymer brushes are a crosslinked network including a plurality of crosslinks, wherein the crosslinks are composed of ethylene glycol dimethacrylate (EGDMA), triethylene glycol dimethacrylate (TEGDMA), trimesic acid, itaconic acid, or combinations thereof,
contacting a medium including two or more organic solvents with the membrane.

16. The method according to claim 15, wherein the mol. % crosslinker in the polymer brush layer is between about 30% and about 70%.

17. The method according to claim 15, wherein the polymer brush layer has a thickness of about 5 nm.

18. The method according to claim 15, wherein contacting a medium including two or more organic solvents with the membrane occurs at a pressure across the membrane greater than about 10 bar.

19. The method according to claim 18, wherein contacting a medium including two or more organic solvents with the membrane occurs at a pressure across the membrane of about 50 bar.

20. The method according to claim 15, wherein the two or more organic solvents include a first solvent including methanol and a second solvent including toluene.
